(19) United States
(12) Patent Application Publication
(10) Pub. No.: US 2022/0184034 A1
(43) Pub. Date: Jun. 16, 2022

(54) METHODS FOR TREATING CUTANEOUS METASTATIC CANCERS

(71) Applicant: Adgero Biopharmaceuticals Holdings, Inc., San Diego, CA (US)

(72) Inventor: Steve Rychnovsky, San Diego, CA (US)

(73) Assignee: Adgero Biopharmaceuticals Holdings, Inc., San Diego, CA (US)

(21) Appl. No.: 17/546,715

(22) Filed: Dec. 9, 2021

Related U.S. Application Data
(60) Provisional application No. 63/123,955, filed on Dec. 10, 2020.

(51) Int. Cl. A61K 31/409; A61K 31/32; A61P 35/04

(52) U.S. Cl. CPC A61K 31/409; A61P 35/04; A61K 31/32

(57) ABSTRACT

A method for treating a cutaneous metastatic cancer comprising administering tin ethyl etiopurprin (SnET2) to a subject suffering from a cutaneous metastatic cancer and exposing the subject at a pre-selected site with light at a wavelength and at a light dose sufficient to effect treatment to effect treatment.

### CROSS-REFERENCE TO RELATED APPLICATION

This application claims the benefit of U.S. application Ser. No. 63/123,955, filed Dec. 10, 2020, expressly incorporated herein by reference in its entirety.

### BACKGROUND

Approximately 5-19% of breast cancer patients suffer from chest wall recurrences after mastectomy. Symptomatic loco-regional breast cancer recurrences in particular have a high impact on physical and psychological well-being and constantly remind patients of the presence of a progressing disease. Cutaneous metastasis breast cancer (CMBC) is a high unmet medical need and remains a therapeutic challenge with few treatment options. Generally, these patients have both CMBC and systemic metastases, both of which may progress despite current interventions. Cutaneously-derived (or associated) symptoms vary in CMBC patients; commonly reported are unrelenting itching and/or pain from these lesions and motion limitation due to discomfort. The lesions often ultimately become confluent, begin to weep and bleed and can become infected, foul smelling and ulcerated masses that lead to very poor quality of life (QoL). If tumor lesions are localized, surgical excision can be attempted. However, these lesions often are widespread throughout the chest wall or involve heavily irradiated tissue. Re-irradiation is generally not indicated to previously radiated lesions due to concerns of additional morbidity from the high doses of radiation required. Such cutaneous metastases also occur with other types of cancer, including other metastatic adenocarcinomas, but typically with less frequency. Therefore, many patients who are radiation refractory will suffer due to these progressing cutaneous tumors, as no other successful local treatment is currently available.

Multiple studies show that photodynamic therapy (PDT) may provide effective tumor control or elimination of primary cutaneous malignancies and have shown the potential of PDT for controlling dermal metastases of breast cancer. PDT is based on photo-oxidation induced selective tumor destruction. The process involves the administration of a photosensitizing drug that is retained by tumor cells and tumor vasculature followed later by local light illumination to activate the photosensitizing drug.

In the treatment of cutaneous cancers including CMBC, light of sufficient wavelength and dose (energy per unit area) is topically delivered, such as with lasers using fiber-optic light delivery devices to shine activating light on the skin surface. This light activates the photosensitizing drug which then acts as a catalyst to generate highly reactive oxygen intermediates that provide the mechanism of action. These intermediates irreversibly oxidize essential cellular components. The resultant photodestruction of crucial cell organelles and vasculature ultimately causes cell death via apoptosis, necrosis and vascular occlusion. Treatment related adverse effects most frequently cited in prior clinical trials were body pain and photosensitivity.

In the case breast cancer, since 2012, more than 230,000 women have been diagnosed with breast cancer in the US every year; nearly 253,000 were diagnosed in 2017. Although significant progress has been achieved in treating breast cancer, metastatic breast cancer is still an incurable disease. Approximately 40,000 women die from breast cancer every year. The focus of treatment of metastatic breast cancer is on controlling the disease for as long as possible while maintaining an acceptable quality of life. In the case of hormone receptor positive (HR positive) metastatic breast cancer, treatment is primarily based on anti-estrogen strategies. However, the majority of patients with metastatic disease will have disease progression due to endocrine resistance. Such patients will then require chemotherapy for control of the cancer and palliation of symptoms from the disease. Similarly, in HER2-positive breast cancer, the majority of patients with metastatic disease experience disease progression following first or second line HER2-targeted therapies and will require the use of chemotherapy. In the case of triple negative breast cancer (TNBC) no such targeted therapies exist and treatment options are more limited.

Over the last two decades, anthracycline and cyclophosphamide have been used in early-stage breast cancer patients as an adjuvant therapy. More recently, taxanes have emerged as another important chemotherapy option in adjuvant therapy of breast cancer. Patients who experience progression of disease on these standard chemotherapy medications have several other chemotherapy options. These include any drug therapy approved for use in the underlying cancer, including but not limited to eribulin, capecitabine, vinorelbine, gemcitabine, carboplatin, or ixabepilone.

The American Society of Clinical Oncology guidelines endorse the use of sequential single agent chemotherapy in patients with metastatic breast cancer except in a clinical setting of impending visceral crisis where combination chemotherapy may generally be preferred. The use of any of these chemotherapy options are considered acceptable and is usually determined by the physician's choice or toxicity profile of the medication.

Despite the advances in the treatment of metastatic breast cancer, a need exists for additional therapies, including combination therapies, to improve therapeutic outcomes. The present invention seeks to fulfill this need and provide further related advantages.

### SUMMARY

The present invention provides methods for treating cutaneous metastatic cancers and the use of tin ethyl etiopurpurin as a photosensitizer in a photodynamic therapeutic treatment of cutaneous metastatic cancers.

In one aspect, the invention provides a method for treating a cutaneous metastatic cancer (e.g., a cutaneous metastatic adenocarcinoma). In certain embodiments, the method comprises:

(a) administering tin ethyl etiopurpurin (SnET2) at a dose from about 0.5 to about 1.0 mg/kg to a subject suffering from a cutaneous metastatic cancer, and

(b) exposing the subject at a pre-selected site with light at a wavelength and at a light dose sufficient to effect treatment.

In certain embodiments of the above method, the subject has been or is being treated with a chemotherapeutic agent selected from the group consisting of eribulin, capecitabine, gemcitabine, vinorelbine, and taxanes (e.g., docetaxel, paclitaxel).

In certain embodiments of the above methods, SnET2 is administered at a dose of about 0.8 mg/kg. In certain embodiments of the above methods, SnET2 is administered at a dose less than about 0.8 mg/kg. In certain embodiments of the above methods, SnET2 is administered at a dose between 0.8 and 1.0 mg/kg.

In a related aspect, the invention provides a method for treating a cutaneous metastatic cancer, comprising:

(a) administering tin ethyl etiopurpurin (SnET2) at a dose from about 0.5 to about 1.0 mg/kg to a subject suffering from a cutaneous metastatic cancer and is receiving Treatment Physician's Choice system therapy, and

(b) exposing the subject at a pre-selected site with light at a wavelength and at a light dose sufficient to effect treatment.

In certain embodiments of the above method, SnET2 is administered at a dose from about 0.5 to about 0.8 mg/kg. In other embodiments, SnET2 is administered at a dose of about 1.0 mg/kg. In other embodiments, SnET2 is administered at a dose of about 0.8 mg/kg.

In certain embodiments of the above method, the Treatment Physician's Choice system therapy is a chemotherapy. In certain of these embodiments, the Treatment Physician's Choice system therapy comprises administration of a chemotherapeutic agent selected from the group consisting of eribulin, capecitabine, gemcitabine, vinorelbine, and taxanes (e.g., docetaxel, paclitaxel).

In certain embodiments of the above methods, the cutaneous metastatic cancer is a cutaneous metastatic adenocarcinoma. Representative cutaneous metastatic adenocarcinomas treatable by the methods of the invention include cutaneous metastatic breast cancer, cutaneous metastatic colon cancer, cutaneous metastatic colorectal cancer, cutaneous metastatic lung cancer, and cutaneous metastatic head and neck cancers. In one embodiment, the cutaneous metastatic adenocarcinoma is cutaneous metastatic breast cancer.

In other embodiments of the above methods, the cutaneous metastatic cancer is superficial inflammatory breast cancer, cutaneous T-cell lymphoma, neuroendocrine tumors, or melanoma metastases.

In certain embodiments of the above methods, the subject is refractive to or not amenable to radiotherapy. In other embodiments, the subject is one where surgery is not indicated.

In certain embodiments of the above methods, the subject is HR positive/HER2 negative and refractive toward endocrine therapy.

In other embodiments of the above methods, the subject is HER2 positive and has failed trastuzumab (HERCEPTIN®)±pertuzumab (PERJETA®) and ado-trastuzumab emtansine (KADCYLA®) treatment regimens.

In certain embodiments of the above methods, SnET2 is administered intravenously at a rate of about 2 mL/kg/hr as an SnET2 emulsion formulation having an SnET2 concentration of about 1.0 mg/mL.

In certain embodiments of the above methods, exposing the subject at a pre-selected site with light at a wavelength and at a light dose sufficient to effect treatment comprises initial light treatment about 12 to about 72 hours post-administration of SnET2. In certain embodiments, the light at a wavelength and at a light dose to effect treatment is delivered by a diode laser light source. In certain embodiments, the wavelength to effect treatment is from about 660 to about 680 nm. In certain embodiments, the wavelength sufficient to effect treatment is about 664 nm (e.g., ±7 nm).

In certain embodiments of the above methods, the light at a wavelength and at a light dose sufficient to effect treatment is delivered by a laser having a power density at the treatment site of about 50 mW/cm² to about 300 mW/cm². In other embodiments, the light of a wavelength sufficient to effect treatment is delivered by a laser having a power density at the treatment site of about 50 mW/cm² to about 150 mW/cm². In further embodiments, the light of a wavelength sufficient to effect treatment is delivered by a laser having a power density at the treatment site of about 150 mW/cm².

In certain embodiments of the above methods, the light at a wavelength and at a light dose sufficient to effect treatment is delivered at a light dose from about 100 J/cm² per lesion to about 200 J/cm² per lesion. In other embodiments, the light of a wavelength sufficient to effect treatment is delivered at a light dose at about 100 J/cm² per lesion.

In certain embodiments of the above methods, the methods further comprise preventing the light from reaching normal skin or lesions previously treated to avoid overexposure of light.

In certain embodiments of the above methods, the methods further comprise actively cooling the subject's skin for irradiance levels above 200 mW/cm².

### DETAILED DESCRIPTION

The present invention provides methods for treating cutaneous metastatic cancers and the use of tin ethyl etiopurpurin as a photosensitizer in a photodynamic therapeutic treatment of cutaneous metastatic cancers.

The methods described herein are photodynamic therapeutic methods that utilize a photosensitizer as an active agent accumulated at the site of treatment. In the methods, light of sufficient wavelength and dose (energy per unit area) is topically delivered, such as with lasers using fiber-optic light delivery devices to shine activating light on the skin surface (i.e., irradiate the site of treatment with accumulated photosensitizer).

In one aspect, the invention provides methods for treating a cutaneous metastatic cancer (e.g., a cutaneous metastatic adenocarcinoma).

In one embodiment, the method comprises:

(a) administering tin ethyl etiopurpurin (SnET2) at a dose from about 0.5 to about 1.0 mg/kg to a subject suffering from a cutaneous metastatic cancer, and

(b) exposing the subject at a pre-selected site with light at a wavelength and at a light dose sufficient to effect treatment.

In certain of these embodiments, SnET2 is administered at a dose of about 0.8 mg/kg. In other of these embodiments, SnET2 is administered at a dose less than about 0.8 mg/kg. In certain of these embodiments, SnET2 is administered at a dose from about 0.8 mg/kg to about 1.0 mg/kg.

In another embodiment, the method comprises:

(a) administering tin ethyl etiopurpurin (SnET2) at a dose from about 0.5 to about 1.0 mg/kg to a subject suffering from a cutaneous metastatic cancer and receiving Treatment Physician's Choice system therapy, and

(b) exposing the subject at a pre-selected site with light at a wavelength and at a light dose sufficient to effect treatment.

In certain of these embodiments, SnET2 is administered at a dose from about 0.8 to about 1.0 mg/kg. In other of these embodiments, SnET2 is administered at a dose from about 0.5 to about 0.8 mg/kg. In further of these embodiments, SnET2 is administered at a dose of about 0.8 mg/kg or at a dose of about 1.0 mg/kg.

In the above methods, the pre-selected site is the site of treatment for cutaneous metastatic cancers (i.e., patient's lesions).

Initial photosensitizer (i.e., SnET2) dose range studies on subjects with various types of lesions indicated a drug dose threshold of about 0.5 to about 0.8 mg/kg (i.e., below that no response was observed and at that level and greater (1.2 mg/kg) good responses were observed. The initial studies did not indicate significant differences for light doses from 100 J/cm² to 200 J/cm² or with treatment timepoints ranging from 24 to 72 hours post administration.

Subsequent studies were conducted with dose parameters of 1.2 mg/kg of photosensitizer, 200 J/cm² of light, and 24 hours post administration as a treatment timepoint. In these studies, slow healing following treatment was observed.

As described herein, advantageous short-term healing and long-term efficacy are achievable at a lower photosensitizer dose, such as 0.8 mg/kg. In certain embodiments of the methods described herein, the photosensitizer dose is from about 0.5 mg/kg to about 1.0 mg/kg. In other embodiments, the photosensitizer dose is about 0.5-0.8 mg/kg. In further embodiments, the photosensitizer dose is about 0.8 mg/kg or about 1.0 mg/kg.

It will be appreciated that in the methods described herein, the photosensitizer dose and the light dose may be varied to achieve optimal results. However, for the reasons set forth above, the methods of the invention do not include a photosensitizer dose of 1.2 mg/kg coupled with a light dose of 200 J/cm².

In certain embodiments of the above methods, the cutaneous metastatic cancer is a cutaneous metastatic adenocarcinoma. Representative cutaneous metastatic adenocarcinomas treatable by the methods of the invention include cutaneous metastatic breast cancer, cutaneous metastatic colon cancer, cutaneous metastatic colorectal cancer, cutaneous metastatic lung cancer, and cutaneous metastatic head and neck cancers. In one embodiment, the cutaneous metastatic adenocarcinoma is cutaneous metastatic breast cancer.

In other embodiments of the above methods, the cutaneous metastatic cancer is superficial inflammatory breast cancer, cutaneous T-cell lymphoma, neuroendocrine tumors, or melanoma metastases.

The lesions of the cutaneous metastatic cancers treatable by the methods described herein are clinically indistinguishable making the methods useful for the treatment of a variety of cutaneous cancers.

In the methods, the photosensitizer, tin ethyl etiopurpurin (SnET2), is administered to the subject to be treated. SnET2 is a synthetic chlorin with the molecular formula of C₃₇H₄₂Cl₂N₄O₂Sn and a molecular weight of 764.4 grams/mole. The chemical structure of SnET2 is shown below.

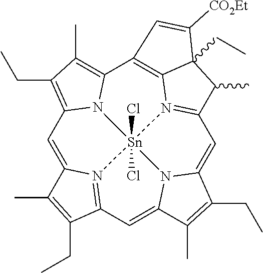

SnET2 is a racemic mixture of two photoactive enantiomers, with two centers of asymmetry at C-18 and C-19. NMR spectroscopy and single crystal x-ray analysis indicate that two enantiomers (18R, 19S and 18S, 19R) are present and chiral chromatography confirms the presence of the two enantiomers in approximately equal proportions. Optical rotation data also indicate a mixture of equivalent amounts of two enantiomers.

In the practice of the methods, SnET2 is administered intravenously as an emulsion formulation. In certain embodiments, the emulsion formulation is a sterile, hydrophobic, isotonic, iso-osmotic lipid emulsion for intravenous infusion into humans. Emulsion formulations useful in the methods of the invention are described U.S. Pat. No. 5,616,342, expressly incorporated herein by reference in its entirety.

In certain embodiments, representative emulsion formulations suitable for administering a poorly water-soluble, pharmacologically active, photosensitizing compound (e.g., SnET2) comprise a pharmacologically acceptable lipoid as a hydrophilic phase, an effective amount of a photoreactive compound, a surfactant, and a cosurfactant. In certain of these embodiments, the cosurfactant is a salt of a bile acid selected from the group of cholic acid, deoxycholic acid, glycocholic acid, and mixtures thereof. Representative emulsion formulations are not liposomal formulations.

Suitable hydrophobic components (lipidoids) comprise a pharmaceutically acceptable triglyceride, such as an oil or fat of a vegetable or animal nature, and preferably is selected from the group consisting of soybean oil, safflower oil, marine oil, black currant seed oil, borage oil, palm kernel oil, cotton seed oil, com oil, sunflower seed oil, olive oil or coconut oil. Physical mixtures of oils and/or inter-esterified mixtures can be employed, if desired. The preferred oils are medium chain length triglycerides having C8-C10 chain length and more preferably being saturated. The preferred triglyceride is a distillate obtained from coconut oil. The emulsion usually has a fat or oil content of about 5 to about 50 g/100 mL, preferably about 10 to about 30 g/100 mL, a typical example being about 20 g/100 mL of the emulsion.

The emulsion may include a stabilizer such as phosphatides, soybean phospholipids, non-ionic block copolymers of polyoxyethylene and polyoxypropylene (e.g., poloxamers), synthetic or semi-synthetic phospholipids, and the like. A preferred stabilizer is purified egg yolk phospholipid. The stabilizer is usually present in the composition in amounts of about 0.1 to about 10, and preferably about 0.3 to about 3 grams/100 mL, a typical example being about 1.5 grams/100 mL.

In certain embodiments, the emulsion advantageously includes a bile acid salt as a co-stabilizer. The salts are pharmacologically acceptable salts of bile acids selected from the group of cholic acid, deoxycholic acid, and glycocholic acid, and preferably of cholic acid. The salts are typically alkaline metal or alkaline earth metal salts and preferably sodium, potassium, calcium or magnesium salts, and most preferably, sodium salts. Mixtures of bile acid salts can be employed if desired. The amount of bile acid salt employed is usually about 0.01 to about 1.0 and preferably about 0.05 to about 0.4 grams/100 mL, a typical example being about 0.2 grams/100 mL.

The emulsion typically has a pH of about 7.5 to about 9.5, and preferably about 8.5. The pH can be adjusted to the desired value, if necessary, by adding a pharmaceutically acceptable base, such as sodium hydroxide, potassium hydroxide, calcium hydroxide, magnesium hydroxide, and ammonium hydroxide.

The emulsion also includes water for injection in the necessary amount to provide the desired volume. If desired, the emulsion can include auxiliary ingredients, such as auxiliary surfactants, isotonic agents, antioxidants, nutritive agents, trace elements, and vitamins.

In certain embodiments of the emulsion formulation, the amount of said photosensitizing compound is about 0.01 to about 1 g/100 mL, the amount of said lipoid is about 5 to about 40 g/100 mL, and the amount of said salt of a bile acid is about 0.05 to about 0.4 g/100 mL.

In certain of these embodiments, the emulsion formulation includes a pharmacologically acceptable lipid as a hydrophobic phase dispersed in a hydrophilic phase, an effective amount of a photosensitizer (e.g., SnET2), a phospholipids stabilizer, and as a co-stabilizer, a pharmaceutically acceptable salt of a bile acid selected from the group consisting of cholic acid, deoxycholic acid, glycocholic acid; and mixtures thereof, in which the concentration of said pharmaceutically acceptable salt is about 0.01 to about 1.0 g/100 mL of the emulsion.

A representative SnET2 emulsion formulation useful in the methods is summarized in Table 1.

TABLE 1
Composition of a representative SnET2
emulsion for intravenous infusion.

| | Reference to | Quantity | |
|---|---|---|---|
| Component | Quality Std | (per mL¹) | Function |
| Tin ethyl etiopurpurin | — | 1.05 mg/mL | Active |
| (SnET2) | | | Pharmaceutical |
| | | | Ingredient |
| Miglyol oil 810 (Medium | USP/Ph. Eur | 200 mg/mL | Vehicle |
| Chain Triglycerides) | | | |
| Egg Yolk Phospholipid | — | 15 mg/mL | Emulsifier |
| Sodium Cholate | — | 2 mg/mL | Co-surfactant |
| Tocopherol | USP | 1 mg/mL | Anti-oxidant |
| Ethanol | USP | 10 mg/mL | Solvent |
| Glycerin | USP | 19 mg/mL | Tonicity Agent |
| Sodium Hydroxide | NF | 3.8 ul | pH Adjustment |
| Water for Injection | USP | qs to 1 mL | Aqueous Solvent |

¹Dose Injection Volume (based on dose of 1.2 mg/kg)
Note:
Nitrogen (gas), NF is used to maintain an inert atmosphere during the manufacturing of each batch.
NF = National Formulary
USP =United States Pharmacopeia
Ph. Eur. = European Pharmacopoeia

As noted above, in the methods of the invention, tin ethyl etiopurpurin (SnET2) is administered intravenously as a lipid emulsion. In certain of these embodiments, SnET2 is administered at a rate of about 2 mL/kg/hr as an SnET2 emulsion formulation having an SnET2 concentration of about 1.0 mg/mL.

In the practice of the methods of the invention, once SnET2 has been administered, the subject is exposed at a pre-selected site (cancerous lesion) with light of a wavelength and light dose to effect treatment comprises initial light treatment about 12 to about 72 hours (e.g., 24±2 hours) post-administration of SnET2. Typically, a patient is infused one day and light treatment is the following day. In certain embodiments, the retreatment interval is about 3 months.

In the methods, the light of a wavelength (or wavelength band) sufficient to effect treatment is effective to activate the photosensitizer SnET2. It will be appreciated that the wavelength of light is a band of wavelengths centered about a wavelength. Although lasers and diodes are identified as providing light at a specific wavelength, the light is delivered at as a wavelength band (e.g., narrow band centered around a specified wavelength).

When SnET2 is the photosensitizer, the wavelength is in the range from about 640 to about 680 nm (e.g., about 665 nm). In certain embodiments, the wavelength sufficient is from about 660 nm to about 680 nm. In a representative embodiment, the wavelength is about 664 nm (e.g., ±7 nm), for example, 665 nm (e.g., ±5 nm). In certain embodiments, the light sufficient to effect treatment is delivered by a diode laser light source.

In certain of embodiments, the laser has a power density at the treatment site of about 50 mW/cm² to about 300 mW/cm². In other of these embodiments, the laser has a power density at the treatment site of about 50 mW/cm² to about 150 mW/cm². In a further embodiment, the laser has a power density at the treatment site of about 150 mW/cm². As used herein, the term “power density” (also known as “irradiance”) is defined as the power (mW) delivered to the tissue divided by the area (cm² of the tissue being irradiated. Power density (mW/cm²) is calculated by dividing light dose (J/cm² by treatment duration(s).

In certain of these embodiments, the light is delivered at a light dose from about 100 J/cm² per lesion to about 200 J/cm² per lesion. In certain embodiments, the light dose is about 100 J/cm² per lesion. In other embodiments, the light dose is about 200 J/cm² per lesion. In further embodiments, the light dose is about 150 J/cm² per lesion. As used herein, the term “light dose” refers to the total amount of energy given per unit area of surface treated. Light dose (J/cm²) is calculated by multiplying power density (W/cm²) by treatment duration(s).

Suitable devices for delivering the light in the methods described herein include laser and light emitting diodes, and preferably diode-based laser devices. In one embodiment, the device includes an embedded diode laser (e.g., 5W) coupled in an optical fiber (e.g., 400 μm) and delivers light at 665±5 nm (90% of spectral power between 660 and 670 nm). The representative device delivers light as a substantially circular spot having an adjustable diameter from about 1.0 to about 6.0 cm with irradiance at target (therapy beam) of 150 mW/cm² for all spots. The device may include an aiming beam (e.g., 532 ±20 nm).

To limit potential side reaction to normal skin, in certain embodiments the method further includes preventing the light from reaching normal skin. In one embodiment, preventing the light from reaching normal skin includes putting a drape over the patient's lesions fields. For example, for a series of lesions in a line 5 cm long, treatment includes irradiation with a circular light field about 6 cm in diameter and results in normal skin being exposed. In another embodiment, preventing the light from reaching normal skin includes putting a drape on the patient with an opening cut generally to the shape of the lesion field. In a further embodiment, preventing the light from reaching normal skin includes applying a light-scattering (or absorbing) composition (e.g., grease) over the normal skin so that the light is prevented from reaching the normal skin. A representative light-scattering composition is a zinc oxide and a representative light absorbing material is a carbon black composition.

To further limit potential side reaction to normal skin, in certain embodiments the method includes actively cooling patient's skin for irradiance levels above 200 mW/cm².

In certain embodiments of the methods described herein, the method is a combination therapy: in addition to photodynamic therapeutic treatment using tin ethyl etiopurpurin (SnET2), the subject is also receiving another therapy, such as a chemotherapy or radiation therapy. In certain of these embodiments the other therapy is a Treatment Physician's Choice system therapy. In these embodiments, the Treatment Physician's Choice system therapy is chemotherapy (i.e., the administration of a chemotherapeutic agent) that is known to be effective for treating (e.g., approved by the FDA for the treatment of) cutaneous metastatic cancer, such as cutaneous metastatic breast cancer (CMBC).

Most oncologists would consider capecitabine as an ideal chemotherapy choice in a patient with endocrine resistant hormone receptor-positive metastatic breast cancer with predominantly skeletal disease especially due to the fact that it is an oral alternative to other intravenous options. Eribulin is an active agent that has been studied in a large randomized phase III trial called the EMBRACE trial. Patients in the EMBRACE trial were randomized to getting either study drug (eribulin) or any chemotherapy of physician's choice. The study showed that eribulin is an active chemotherapy drug in metastatic breast cancer. Interestingly, it showed that vinorelbine, gemcitabine, capecitabine and taxanes were the most common choices in the control arm of the study. The three taxane options—nab-paclitaxel, paclitaxel and docetaxel—have all been studied against each other. Generally, all three medications are considered interchangeable as they have similar efficacy, but with different side effect profiles.

It is imperative to balance the need between providing appropriate treatment options for a physician to choose from and reducing the likelihood of side effect modification of the trial intervention from these medications. In this regard, five chemotherapy options are provided for treating physician's—eribulin, capecitabine, taxanes, gemcitabine and vinorelbine—to provide sufficient flexibility to ensure appropriate metastatic breast cancer (MBC) management, and to provide for sufficient understanding of any confounding impacts on effectiveness.

The following describes examples of approved Treatment Physician's Choice system therapies for patients with metastatic breast cancer, including cutaneous metastatic breast cancer.

HALAVEN® (eribulin mesylate) injection. Eribulin is a mechanistically unique inhibitor of microtubule dynamics, binding predominantly to a small number of high affinity sites at the plus ends of existing microtubules. Eribulin has both cytotoxic and non-cytotoxic mechanisms of action. Its cytotoxic effects are related to its antimitotic activities, wherein apoptosis of cancer cells is induced following prolonged and irreversible mitotic blockade.

XELODA® (capecitabine) tablets. Capecitabine is a chemotherapeutic agent that acts as an anti-metabolite. Capecitabine administration results in the transformation of capecitabine to fluorouracil, a common chemotherapeutic agent that prevents cells from making and repairing DNA as required by cancer cells for growth and proliferation.

GEMZAR® (Gemcitabine) for injection. Gemcitabine is a chemotherapeutic agent that inhibits thymidylate synthetase, leading to inhibition of DNA synthesis and cell death. Gemcitabine is a prodrug and its activity results from intracellular conversion by deoxycitidine kinase to two active metabolites, gemcitabine diphosphate and gemcitabine triphosphate.

TAXOTERE® (docetaxel injection); ABRAXANE® (nab-paclitaxel); and TAXOL® (paclitaxel injection). Taxanes are among the first line of treatments for breast cancer. Paclitaxel is a chemotherapeutic agent of the taxane family Paclitaxel binds to cells in a specific and saturable manner with a single set of high-affinity binding sites. The microtubule cytoskeleton is reorganized in the presence of paclitaxel and extensive parallel arrays or stable bundles of microtubules are formed in cells growing in tissue culture. Paclitaxel blocks cells in the G2/M phase of the cell cycle and such cells are unable to form a normal mitotic apparatus.

Docetaxel is a second-generation chemotherapeutic agent of the taxane family A derivative of paclitaxel, the first taxane to hit the market, docetaxel's primary mechanism of action is to bind beta-tubulin, enhancing its proliferation and stabilizing its conformation. Doing so inhibits the proper assembly of microtubules into the mitotic spindle, arresting the cell cycling during G2/M. Docetaxel also reduces the expression of the bcl-2 gene, an anti-apoptotic gene often overexpressed by cancer cells conferring enhanced survival. By downregulating this gene, tumor cells can more readily undergo apoptosis. Thus, docetaxel is believed to have a twofold mechanism of antineoplastic activity: (1) inhibition of microtubular depolymerization, and (2) attenuation of the effects of bcl-2 and bcl-xL gene expression. Taxane-induced microtubule stabilization arrests cells in the G2/M phase of the cell cycle and induces bcl-2 phosphorylation, thereby promoting a cascade of events that ultimately leads to apoptotic cell death.

NAVELBINE® (vinorelbine tartare). Vinorelbine is a vinca-alkaloid with a broad spectrum of anti-tumor activity. Vinca-alkaloids are categorized as spindle poisons, and their mechanism of action is to interfere with the polymerization of tubulin, a protein responsible for building the microtubule system that appears during cell division.

Vinorelbine is a mitotic spindle poison that impairs chromosomal segregation during mitosis. Vinorelbine binds to microtubules and prevents formation of the mitotic spindle, resulting in the arrest of tumor cell growth in the G2/M phase of the cell cycle.

Because the mechanism of actions for the Treatment Physician's Choice system therapies for cutaneous metastatic breast cancer noted above are independent and distinct from photodynamic therapeutic treatment using tin ethyl etiopurpurin (SnET2), interference resulting in therapeutic ineffectiveness or adverse side effects due to interaction of tin ethyl etiopurpurin (SnET2) with any one of the Treatment Physician's Choice system therapies for cutaneous metastatic breast cancer noted above is not expected.

Therefore, in embodiments where the cutaneous metastatic cancer is cutaneous metastatic breast cancer, the Treatment Physician's Choice system therapy comprises administration of a chemotherapeutic agent selected from the group consisting of eribulin, capecitabine, gemcitabine, vinorelbine, and taxanes (e.g., docetaxel, paclitaxel).

In certain embodiments, the invention provides method for treating a cutaneous metastatic cancer, comprising treating a subject suffering from a cutaneous metastatic cancer with a combination of a phototherapeutic treatment with tin ethyl etiopurpurin (SnET2) as described herein and a chemotherapeutic agent selected from the group consisting of eribulin, capecitabine, gemcitabine, vinorelbine, and a taxane (e.g., docetaxel, paclitaxel).

Approved Treatment Physician's Choice system therapies for patients with metastatic breast cancer, including cutaneous metastatic breast cancer, also include radiation therapies.

Subjects suitable for treatment by the methods of the invention include those refractive toward or not amenable to radiotherapy; those where surgery is not indicated; those that are HR positive/HER2 negative and refractive toward endocrine therapy; or those that are HER2 positive and have failed trastuzumab (HERCEPTIN®)±pertuzumab (PERJETA®) and ado-trastuzumab emtansine (KADCYLA®) treatment regimens.

The following is a description of a representative method of the invention for treating cutaneous metastatic breast cancer.

As described herein, in one embodiment, the invention provides a method for treating cutaneous metastatic breast cancer using SnET2 photodynamic therapy (PDT), which involves a laser light source, a fiber-optic light delivery device, and the photosensitizer SnET2. In this representative method, SnET2 is supplied as an emulsion formulation at a concentration of 1.0 mg/ml, suitable for parenteral use in single-use 20 mL glass vials. SnET2 is administered to patients with symptomatic cutaneous metastatic breast cancer. In certain embodiments, SnET2 is administered to patients who have been or who are being treated with Treatment Physician's Choice (TPC) systemic therapy.

Dosage, Administration, and Schedule

Patients are administered SnET2 at a dose as described herein (e.g., 0.5-1.0 mg/kg, 0.5-0.8 mg/kg, 0.8-1.0 mg/kg, 0.8 mg/kg, or 1.0 mg/kg) by intravenous injection, for example, at a rate of 2 mL/kg/hr.

The subjects receive the light treatment the following day (e.g., 24±2 hours) post-infusion of the photosensitizer SnET2. The light treatment is applied with a diode laser light source that emits light at about 664 nm (e.g., 664±7 nm), for example, 665±5 nm. The light treatment is performed according to the following light dosimetry according to the appropriate dosimetry tables:
delivered power density at the skin surface of 50 mW/cm² to 150 mW/cm²
light dose 100-200 J/cm² per lesion (e.g., 100 or 200 J/cm² per lesion)
treatment of lesions is by surface (non-contact) illumination delivered by a microlens fiberoptic light delivery probe or similar device that provides equivalent illumination such as light that is spatially uniform (within +/−33% of average irradiance) at the treatment site equivalent
maximum light treatment field diameter is restricted to 6 cm (corresponding to 28.28 cm²), the treatment light field should extend no more than 0.5 cm beyond the longest dimension of the lesion field to be treated
individual light treatment fields must be separated by at least 1 cm.

Clinical Dosimetry

Two Phase 1/2 studies established the clinical dosimetry. The first study was a dose escalation study that enrolled a total of 22 patients with cutaneous lesions arising from either basal cell cancer, squamous cell cancer or CMBC. A total of 213 lesions were treated using photosensitizer SnET2) doses ranging from 0.1-1.2 mg/kg, light doses of 100, 150 or 200 J/cm² and treatment timepoints of 24, 48, or 72 hours post-infusion. Two key measures were utilized in these studies, lesion reaction and lesion response. Lesion reaction was an acute characterization of the treatment effect that was performed out to 1 week post-treatment according to the following scale:
Grade 0=No visual erythema or edema
Grade 1=Faint erythema and/or slight edema
Grade 2=Moderate erythema and edema
Grade 3=Severe discoloration, edema, sloughing or eschar

Beginning at one month post-treatment and then at 3 and 6 months post-treatment, a lesion response assessment was conducted according to the following scoring system:
Complete Response (complete reduction of the lesion)
Partial Response (more than 50% reduction of the lesion)
Failure (less than 50% reduction of the lesion).

The study results show minimal variation in response with treatment timepoint, i.e., response rates were similar when the light was given at 24, 48, or 72 hours after administration of the photosensitizer. Therefore, in the analysis that follows, results from all three treatment timepoints have been combined.

In terms of individual lesion response, Table 2 shows the number of lesions treated at each dose combination (photosensitizer doses ranging from 0.1-1.2 mg/kg, light doses of 100, 150 or 200 J/cm²).

TABLE 2
Individual Lesion Responses.

| Drug/Light | Total | Partial | Complete | | |
|---|---|---|---|---|---|
| Dose | Lesions | Res- | Res- | Total | Response |
| Combination | Treated | ponders | ponders | Responders | Rate (%) |
| 0.1/100 | 20 | 0 | 0 | 0 | 0 |
| 0.1/150 | 22 | 0 | 0 | 0 | 0 |
| 0.1/200 | 19 | 0 | 0 | 0 | 0 |
| 0.2/100 | 2 | 1 | 0 | 1 | 50 |
| 0.2/150 | 2 | 0 | 0 | 0 | 0 |
| 0.2/200 | 3 | 0 | 0 | 0 | 0 |
| 0.4/100 | 13 | 0 | 0 | 0 | 0 |
| 0.4/150 | 15 | 0 | 0 | 0 | 0 |
| 0.4/200 | 15 | 0 | 0 | 0 | 0 |
| 0.8/100 | 14 | 2 | 9 | 11 | 78 |
| 0.8/150 | 15 | 5 | 8 | 13 | 87 |
| 0.8/200 | 15 | 1 | 12 | 13 | 87 |
| 1.0/100 | 3 | 0 | 3 | 3 | 100 |
| 1.0/150 | 5 | 1 | 4 | 5 | 100 |
| 1.0/200 | 8 | 0 | 8 | 8 | 100 |
| 1.2/100 | 12 | 7 | 5 | 12 | 100 |
| 1.2/150 | 16 | 9 | 7 | 16 | 100 |
| 1.2/200 | 14 | 3 | 11 | 14 | 100 |

The results of this study show a threshold at the 0.8 mg/kg dose. Only 1 of 111 lesions treated at doses less than 0.8 mg/kg responded while 95 of 102 treated at 0.8 mg/kg or higher had responses.

A similar result was observed when individual patient responses were compared. In these studies, individual patients received a single drug dose and each patient then had individual lesions treated at various light doses (100, 150, or 200 J/cm²) and treatment timepoints (24, 48, 72 hours post-infusion). In Table 3, results for each patient treated at 0.8 mg/kg or higher are provided. In each drug dose case, individual lesions were treated at one of the three light doses and one of the three treatment timepoints, all of which have been combined for each individual patient result in the table.

TABLE 3
Lesion Responses for Individual Subjects.

| Patient | Drug | Lesions | Lesions | % Lesions |
|---|---|---|---|---|
| ID | Dose | Treated | Responded | Responded |
| RC004 | 0.8 | 27 | 20 | 74 |
| WC007 | 0.8 | 9 | 9 | 100 |
| WC008 | 0.8 | 8 | 8 | 100 |
| SC002 | 1 | 6 | 6 | 100 |
| SC003 | 1 | 6 | 6 | 100 |
| SC004 | 1 | 4 | 4 | 100 |
| RC005 | 1.2 | 6 | 6 | 100 |
| RC006 | 1.2 | 1 | 1 | 100 |
| RC007 | 1.2 | 3 | 3 | 100 |
| SC001 | 1.2 | 4 | 4 | 100 |
| SC005 | 1.2 | 6 | 6 | 100 |
| WC009 | 1.2 | 20 | 20 | 100 |
| WC010 | 1.2 | 2 | 2 | 100 |

As these results show, with the exception of one patient dosed at 0.8 mg/kg, all patients treated at a dose of 0.8 mg/kg or higher had a 100% response rate.

A further analysis was undertaken to explore the relationship between the acute lesion reaction and the lesion response. This retrospective examination suggests that the acute lesion reaction may also serve as a useful surrogate in predicting treatment recovery time. Following are the results from patients who were scored for lesion reaction at Week 1 post-treatment and who received a drug dose of 0.8 mg/kg or higher. Referring to Table 4, the column on the far-right side (% with Max Rx Score at Week 1) shows the percentage of lesions treated at the corresponding dose combination that received the maximum lesion reaction grade at Week 1 post-treatment (the last timepoint at which lesion reaction was measured). For the 0.8 mg/kg cases, only 5 of the 37 lesions (14%) received the highest lesion reaction score. In the case of the 1.2 mg/kg dose group, the percentage of lesions with the maximum lesion reaction score, 13 of 22 (59%), was substantially higher.

TABLE 4
Summary of Percent Responders and Lesion Reaction Scores by Dose.

| | | | | | Total with | % with |
|---|---|---|---|---|---|---|
| | | | | | Max Rx | Max Rx |
| Drug | Light | Total | Total | % | Score at | Score at |
| Dose | Dose | Lesions | Responders | Responders | Week 1 | Week 1 |
| 0.8 | 100 | 14 | 11 | 79 | 0 | 0 |
| 0.8 | 150 | 15 | 13 | 87 | 1 | 7 |
| 0.8 | 200 | 15 | 13 | 87 | 4 | 27 |
| 1.0 | 100 | 3 | 3 | 100 | 0 | 0 |
| 1.0 | 150 | 5 | 5 | 100 | 0 | 0 |
| 1.0 | 200 | 8 | 8 | 100 | 0 | 0 |
| 1.2 | 100 | 6 | 6 | 100 | 2 | 33 |
| 1.2 | 150 | 9 | 9 | 100 | 7 | 78 |
| 1.2 | 200 | 7 | 7 | 100 | 4 | 57 |

Note:
Above data summarizes % responders across lesions, not patients, not accounting for the within patient correlation among lesions.

Delayed Lesion Response Evaluation

A factor that impacts clinical outcome is the time delay required for post-treatment effects to resolve. This factor is observed in the results from four subsequent phase 2/3 studies of CMBC lesions using a fixed drug dose of 1.2 mg/kg and a fixed light dose of 200 J/cm² administered at approximately 24 hours post-infusion. In these studies lesions were first scored for lesion reaction and then scored for lesion treatment response after the lesion reaction had resolved. In the case of lesion reaction scoring, the reaction was first assessed by the investigator at approximately 4-week intervals and assigned a Reaction Score according to the following defined terms:
0=No erythema, edema or residual eschar
1=Mild (faint) erythema and/or slight edema present
2=Moderate erythema and edema present
3=Severe erythema and edema present
4=Sloughing, ulceration, and/or eschar present
5=Eschar requiring debridement

Once a lesion scored a Reaction Score of 0 or 1 it was deemed suitably healed for evaluation, at which time its treatment response was quantified by visual measurement of the two longest dimensions. In those cases where the patient left the study before a reaction score of 0-1 was reached, those lesions were classified as not evaluable.

An analysis conducted of the median time to lesion evaluation (time to reach a reaction score of 0 or 1) in these same four phase 2/3 CMBC trials indicates the time delay for post-treatment effects to resolve was closely associated with the acute (week 1-2 post-treatment) lesion reaction score, with higher acute reaction scores being associated with a longer time to lesion evaluation, consistent with a more severe treatment reaction requiring a longer time to resolve. Table 5 shows the number of lesions evaluated in these four studies as a function of their acute reaction score along with subsequent lesion response rates and median week at which the lesions were evaluable. Because larger lesions are most likely to be clinically relevant, this table only includes those lesions from the same four CMBC phase 2/3 trials that had baseline dimensions larger than 1 cm.

TABLE 5
Summary of Proportion of Lesions with Poor Lesion Reaction Score and
Percentage of Lesions Responding by Acute Lesion Reaction Score.

| | | Total | | | | |
|---|---|---|---|---|---|---|
| Acute | | with Max | | | | Response |
| Lesion | | Reaction | | Median | | Rate of |
| Reaction | | During | Total | Evaluable | | Evaluable |
| Score¹ | Total | Trial ≥4 | Evaluated | Week | Responders | Lesions |
| 0-1 | 93 | 14 | 76 | 1 | 48 | 63% |
| 2-3 | 385 | 165 | 208 | 6 | 168 | 81% |
| 4-5 | 496 | 496 | 204 | 12 | 179 | 88% |

¹See above for lesion reaction scoring scale used in these studies.
Note:
Above statistics based on lesions, not patients as the statistical unit of analysis.

As these results indicate, the median time to evaluation was 12 weeks for acute reactions of 4-5 (eschar or ulceration) versus 6 weeks for lesions with acute lesion reactions of 2-3. However, the difference in lesion response rates in these groups was modest (88% vs 81%).

As used herein, the term “about” refers to ±5% of the specified value.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for treating a cutaneous metastatic cancer, comprising:
(a) administering tin ethyl etiopurpurin (SnET2) at a dose from about 0.5 to about 1.0 mg/kg to a subject suffering from a cutaneous metastatic cancer, and
(b) exposing the subject at a pre-selected site with light at a wavelength and at a light dose sufficient to effect treatment.

2. The method of claim 1, wherein the subject suffering from a cutaneous metastatic cancer and receiving Treatment Physician's Choice system therapy.

3. The method of claim 1, wherein the cutaneous metastatic cancer is a cutaneous metastatic adenocarcinoma.

4. The method of claim 3, wherein the cutaneous metastatic adenocarcinoma is selected from cutaneous metastatic breast cancer, cutaneous metastatic colon cancer, cutaneous metastatic colorectal cancer, cutaneous metastatic lung cancer, and cutaneous metastatic head and neck cancers.

5. The method of claim 1, wherein the cutaneous metastatic cancer is superficial inflammatory breast cancer, cutaneous T-cell lymphoma, neuroendocrine tumors, or melanoma metastases.

6. The method of claim 2, wherein the Treatment Physician's Choice system therapy is a chemotherapy or radiation.

7. The method of claim 2, wherein the Treatment Physician's Choice system therapy comprises administration of a chemotherapeutic agent selected from the group consisting of eribulin, capecitabine, gemcitabine, vinorelbine, and taxanes.

8. The method of claim 1, wherein the subject has been or is being treated with a chemotherapeutic agent selected from the group consisting of eribulin, capecitabine, gemcitabine, vinorelbine, and taxanes.

9. The method of claim 1, wherein the subject is refractive to or not amenable to radiotherapy.

10. The method of claim 1, wherein the subject is one where surgery is not indicated.

11. The method of claim 1, wherein the subject is HR positive/HER2 negative and refractive toward endocrine therapy.

12. The method of claim 1, wherein the subject is HER2 positive and has failed trastuzumab±pertuzumab and ado-trastuzumab emtansine treatment regimens.

13. The method of claim 1, wherein SnET2 is administered at a dose of about 0.8 mg/kg.

14. The method of claim 1, wherein SnET2 is administered at a dose less than about 0.8 mg/kg.

15. The method of claim 1, wherein SnET2 is administered at a dose from about 0.8 to about 1.0 mg/kg.

16. The method of claim 1, wherein SnET2 is administered at a dose from about 0.5 to about 0.8 mg/kg.

17. The method of claim 1, wherein SnET2 is administered at a dose of about 1.0 mg/kg.

18. The method of claim 1, wherein SnET2 is administered intravenously at a rate of about 2 mL/kg/hr as an SnET2 emulsion formulation having an SnET2 concentration of about 1.0 mg/mL.

19. The method of claim 1, wherein exposing the subject at a pre-selected site with light of a wavelength sufficient to effect treatment comprises initial light treatment about 12 to about 72 hours post-administration of SnET2.

20. The method of claim 1, wherein the light of a wavelength sufficient to effect treatment wavelength is delivered by a diode laser light source.

21. The method of claim 1, wherein the wavelength sufficient to effect treatment is from about 660 to about 680 nm.

22. The method of claim 1, wherein the wavelength sufficient to effect treatment is about 665 nm.

23. The method of claim 1, wherein the light of a wavelength sufficient to effect treatment is delivered by a laser having a power density at the treatment site of about 50 mW/cm² to about 300 mW/cm².

24. The method of claim 1, wherein the light of a wavelength sufficient to effect treatment is delivered by a laser having a power density at the treatment site of about 50 mW/cm² to about 150 mW/cm².

25. The method of claim 1, wherein the light of a wavelength sufficient to effect treatment is delivered by a laser having a power density at the treatment site of about 150 mW/cm².

25. The method of claim 1, wherein the light of a wavelength sufficient to effect treatment is delivered at a light dose from about 100 J/cm² per lesion to about 200 J/cm² per lesion.

26. The method of claim 1, wherein the light of a wavelength sufficient to effect treatment is delivered at a light dose at about 100 J/cm² per lesion.

27. The method of claim 1 further comprising preventing the light from reaching normal skin.

28. The method of claim 1 further comprising actively cooling patient's skin for irradiance levels above 200 mW/cm².
